(19) United States
(12) Patent Application Publication
(10) Pub. No.: US 2023/0249790 A1
(43) Pub. Date: Aug. 10, 2023

(54) ROBOTIC FISH ENABLED CARBON DIOXIDE LEAK DETECTION FOR OFFSHORE CARBON DIOXIDE SEQUESTRATION MONITORING

(71) Applicant: UNIVERSITY OF HOUSTON SYSTEM, Houston, TX (US)

(72) Inventors: Zheng Chen, Houston, TX (US); Miao Pan, Houston, TX (US); Jiefu Chen, Houston, TX (US)

(73) Assignee: UNIVERSITY OF HOUSTON SYSTEM, Houston, TX (US)

(21) Appl. No.: 18/105,563

(22) Filed: Feb. 3, 2023

Related U.S. Application Data
(60) Provisional application No. 63/308,315, filed on Feb. 9, 2022.

(51) Int. Cl. B63G 8/00; G01N 31/22; G01N 33/00; G01N 33/18; G06N 3/008; B63G 8/08; G05D 1/00

(52) U.S. Cl. CPC B63G 8/001; B63G 8/08; G01N 31/221; G01N 33/004; G01N 33/0062; G01N 33/1886; G05D 1/0022; G05D 1/0044; G06N 3/008; B63G 2008/004

(57) ABSTRACT

A method for detecting and locating a carbon dioxide leak in a submarine environment includes operating autonomous underwater vehicles (AUVs) in the submarine environment, where the operating includes obtaining, with each AUV of the AUVs, measurements of an attribute indicative of carbon dioxide, communicating, while operating the AUVs, the measurements from the each AUV to other AUVs of the AUVs, mapping, while operating the autonomous AUVs, carbon dioxide concentration in the submarine environment, and guiding the AUVs toward a highest concentration of the carbon dioxide concentration based on the mapping.

### TECHNICAL FIELD

The invention relates in general to the field of identifying carbon dioxide leaks in a submarine environment, and, more specifically, to methods and systems utilizing autonomous underwater vehicles, in particular bio-inspired vehicles, to detect carbon dioxide leaked into a water column and locating the source of the leak.

### BACKGROUND

This section provides background information to facilitate a better understanding of the various aspects of the disclosure. It should be understood that the statements in this section of this document are to be read in this light, and not as admissions of prior art.

Reducing carbon dioxide emission has been a global consensus for mitigating climate change. Recent booming offshore drilling activities have produced a huge amount of oil & gas, the vast majority of which is eventually turned into carbon dioxide through combustion. One of the potentially most effective ways to compensate carbon dioxide emission is to capture carbon dioxide from fossil fuel-burning power plants and store it back to undersea geological formations offshore. It is reported that ExxonMobil is considering Houston, TX for a $100 billion carbon dioxide capture project with carbon dioxide potentially being sequestered in the continental shelf of the Gulf of Mexico. Offshore sequestration could lead to damaging environmental consequences if not handled correctly. Carbon dioxide leakage into the overlying seawater could cause its dissolution, making the seawater more acidic and giving rise to detrimental effects to the marine ecosystem. Furthermore, acidified ocean water can be introduced into coastal estuaries, causing cascading damage to both aquaculture and the natural ecosystem.

### SUMMARY

An exemplary method for detecting and locating a carbon dioxide leak in a submarine environment includes operating autonomous underwater vehicles (AUVs) in the submarine environment, where the operating includes obtaining, with each AUV of the AUVs, measurements of an attribute indicative of carbon dioxide, communicating, while operating the AUVs, the measurements from the each AUV to other AUVs of the AUVs, mapping, while operating the autonomous AUVs, carbon dioxide concentration in the submarine environment, and guiding the AUVs toward a highest concentration of the carbon dioxide concentration based on the mapping.

An exemplary system for detecting and locating a carbon dioxide leak in a submarine environment includes AUVs, each AUV of the AUVs including a sensor to obtain measurements of an attribute in the submarine environment that is indicative of carbon dioxide, a transmitter to communicate the measurements to the each AUV of the AUVs, and a control logic to implement a carbon dioxide gradient estimation using the measurements to calculate a gradient vector, and the system including a swarm logic to guide the AUVs using the gradient vector to a highest carbon dioxide concentration.

This summary is provided to introduce a selection of concepts that are further described below in the detailed description. This summary is not intended to identify key or essential features of the claimed subject matter, nor is it intended to be used as an aid in limiting the scope of claimed subject matter.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion. As will be understood by those skilled in the art with the benefit of this disclosure, elements and arrangements of the various figures can be used together and in configurations not specifically illustrated without departing from the scope of this disclosure.

FIG. 1 illustrates an exemplary system for detecting and locating carbon dioxide leaks in a submarine environment.

FIG. 2 illustrates autonomous underwater vehicles monitoring a submarine environment for a carbon dioxide leak in the vicinity of a carbon dioxide sequestration reservoir.

FIG. 3 illustrates autonomous underwater vehicles guided toward a carbon dioxide leak in a submarine environment.

FIG. 4 illustrates an exemplary autonomous underwater vehicle configured as a bio-inspired robotic fish.

FIG. 5 is a top view illustrating an undulatory body wave of an exemplary bio-inspired robotic fish producing oscillating foil propulsion.

FIG. 6 is a top view of an exemplary bio-inspired robotic fish revealing an exemplary propulsion system in the tail section to produce oscillating foil propulsion.

FIG. 7 illustrates an exemplary double-slider-crank of an exemplary propulsion system of a bio-inspired robotic fish.

FIG. 8 is a block diagram illustrating an exemplary autonomous underwater vehicle.

FIG. 9 illustrates an exemplary method according to aspects of the disclosure.

### DETAILED DESCRIPTION

It is to be understood that the following disclosure provides many different embodiments, or examples, for implementing different features of various illustrative embodiments. Specific examples of components and arrangements are described below to simplify the disclosure. These are, of course, merely examples and are not intended to be limiting. For example, a figure may illustrate an exemplary embodiment with multiple features or combinations of features that are not required in one or more other embodiments and thus a figure may disclose one or more embodiments that have fewer features or a different combination of features than the illustrated embodiment. Embodiments may include some but not all the features illustrated in a figure and some embodiments may combine features illustrated in one figure with features illustrated in another figure. Therefore, combinations of features disclosed in the following detailed description may not be necessary to practice the teachings in the broadest sense and are instead merely to describe particularly representative examples. In addition, the disclosure may repeat reference numerals and/or letters in the various examples. This repetition is for the purpose of simplicity and clarity and does not itself dictate a relationship between the various embodiments and/or configurations discussed.

Reducing carbon dioxide emission has been a global consensus for mitigating climate change, which calls for offshore sequestration to store the carbon dioxide captured from the atmosphere to undersea geological formations. However, offshore sequestration can cause environmental problems since carbon dioxide leakage into the overlying seawater may cause seawater to become more acidic giving rise to detrimental effects to the marine ecosystem. Therefore, autonomous carbon dioxide leakage monitoring and detection technology is an emergent need in offshore carbon dioxide sequestration practice. Aspects of this disclosure include systems and methods for mapping carbon dioxide distribution in seawater using a school of autonomous underwater vehicles, in particular in the form of robotic fish, equipped with highly sensitive pH sensors and/or carbon dioxide sensors; scaling the monitoring range by leveraging the mobility of robotic fish, underwater wireless communication and navigation, and swarming control; and detecting exact leakage location on the seafloor using swarming of bio-inspired robotic fish to the high carbon dioxide concentration.

The ability to detect carbon dioxide leakage from an undersea sequestration site is a forward-looking technology, i.e., before large scale carbon dioxide sequestration takes place, a method for detecting and monitoring the ambient chemical condition should be in place to ensure that carbon dioxide can be effectively preserved to minimize potential environmental impact even if small carbon dioxide leakage occurs. When a carbon dioxide leak occurs, pH (water acidity) in the affected water column will be lower than the unaffected seawater, along with elevated carbon dioxide partial pressure (pCO2). Exemplary subsea leak detection systems and methods use a swarm of bio-inspired autonomous underwater vehicles equipped with highly sensitive pH and carbon dioxide sensors to detect pH and carbon dioxide fields in a wide area of seabed and construct 2-D to Y-D maps of pH and carbon dioxide distribution, for example near the site of carbon dioxide injection. After that, the leakage location can be narrowed using real-time magnetic induction tomography with edge computing.

In general, the methods and systems include three elements, a plurality of autonomous underwater vehicles, in particular in the form of a bio-inspired robotic fish, long and short range wireless communication, and source tracking for the autonomous underwater vehicles to detect the location of the leaking carbon dioxide.

A bio-inspired autonomous robotic fish can be used as an autonomous underwater vehicle (AUV). The robotic fish may integrate a reversible fuel cell enabled buoyancy control for energy efficient and noiseless depth keeping and vertical maneuvering. Compared to traditional AUVs, the exemplary robotic fish has a longer life, better energy efficiency, superior maneuverability in confined environments and shallow waters, and reduced disruption to the environment. The robotic fish may include a novel slot-crank driven tail that can efficiently convert the rotation generated by a DC motor to flapping motion of the tail. A wave-guided model may be used to optimally design the fish tail so that it can mimic the appearance and swimming pattern of real fish and generate high efficiency propulsion.

Long-range and short-range magnetic induction based wireless communication systems, enabling more efficient, accurate, and faster rates of wireless data transfer may be utilized. A subsea Internet of things (IoT) network and corresponding solutions for the sensed information delivery, which leverages the infrastructure of offshore installations and jointly uses satellite, underwater magnetic induction, underwater acoustic and stress wave communications (i.e., wave-guided acoustic signals along an injection pipeline). For robustness, reliability and high availability purposes, the subsea IoT network may enable two paths for the sensed information delivery: 1) robotic fish to float (e.g., SEATREC Profiling Float) and/or offshore infrastructure (e.g., offshore rigs, exploration ships or wind turbines) to a remote-located control center; 2) robotic fish to piezoelectric transducers anchored proximate the seafloor, for example on an offshore pipeline. For both paths, the intra-swarm communications among robotic fish will be via short-range magnetic induction communications.

Source tracking control for robotic fish to detect the location of carbon dioxide leakage may be utilized. A plurality of robotic fish, i.e., swarm or school, will perform pH and/or carbon dioxide mapping. For example, each robotic fish will carry two sensors, one sensor in the head and another sensor in the tail. The sensors may be pH and/or carbon dioxide sensors. The swarm will share the sensed carbon dioxide information through, e.g., magnetic induction based, wireless communication. A partial differential equations-based carbon dioxide gradient estimation will be implemented on each robotic fish so that the gradient vector will be calculated. Based on the gradient vector, a distributed swarming control will guide the robotic fish to swarm toward the center of carbon dioxide leakage.

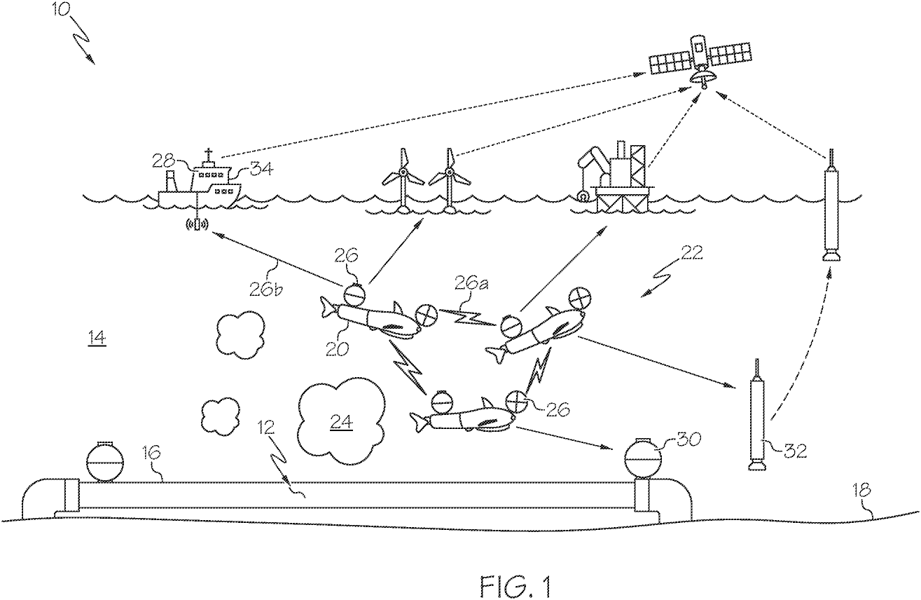

FIG. 1 illustrates an exemplary system 10 of detecting and locating a carbon dioxide leak 12 in a submarine environment 14 (e.g., water column). Leak 12 may be located for example in pipeline 16 or seafloor 18. System 10 utilizes a plurality of autonomous underwater vehicles 20, generally referred to as a swarm or school 22. In the illustrated embodiments, AUVs 20 are in the form of bio-inspired robotic fish. Robotic fish 20 operate over an area of the submarine environment, each of the robotic fish for example monitoring a selected smaller area for indications of increased carbon dioxide 24 in the water column. Each robotic fish includes electronics for measuring attributes indicating carbon dioxide concentration, controlling propulsion of the robotic fish, and communicating with other robotic fish and other locations. For example, each robotic fish 20 includes one or more transmitters 26, e.g., magnetic induction and/or acoustic modem, that can transmit and receive signals from other robotic fish 20, surface installations 28, anchored transmitters 30, and submersible transmitters 32. For example, and without limitation, the AUVs may communicate among themselves via magnetic induction 26a and communicate with other locations via acoustic signals 26b. Submersible transmitters 32 may be submersed to communicate with robotic fish 20 and surface to communicate with surface installations 28, e.g., ships, platforms, satellites, and land sites. A remote-located control center 34 may be located for example at a landsite or surface installation.

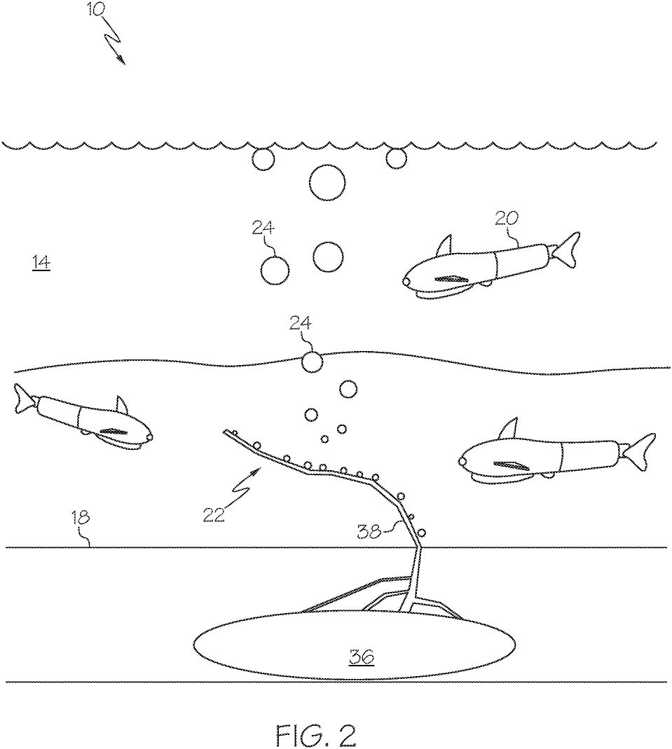

FIG. 2 illustrates carbon dioxide 24 leaking from a subsea carbon dioxide sequestration reservoir 36 through carbon dioxide leak 12, e.g., foundation crack 38 in seafloor 18. Individual AUVs 20 are monitoring for carbon dioxide 24. The AUVs monitor for carbon dioxide 24, for example by measuring acidity with a pH sensor and/or carbon dioxide partial pressure with a carbon dioxide sensor. Each of the AUVs communicates the measured attributes to the other AUVs of the school, with location data. Carbon dioxide concentration in the water column is mapped with the measurements. Each AUV may perform the mapping.

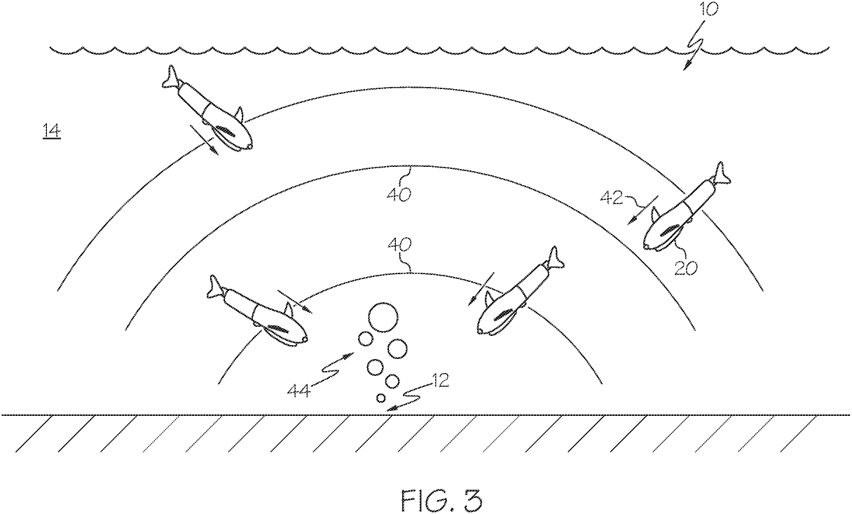

With reference to FIG. 3, the mapping includes implementing a carbon dioxide gradient 40 estimation using the measurements and calculating a gradient vector 42 based on the carbon dioxide gradient estimation. When the measurements indicate carbon dioxide is leaking into the water column, for example carbon dioxide concentration above a normal level or increasing concentration, the school of AUVs 20 are guided toward a highest concentration 44 of carbon dioxide based on the mapping. Guiding the plurality of AUVs 20 toward the highest concentration may be initiated from the remote-located control center or via instructions on each AUV. The measurements and mapping continue as the AUVs are guided toward the highest concentration thereby locating the position of the carbon dioxide leak.

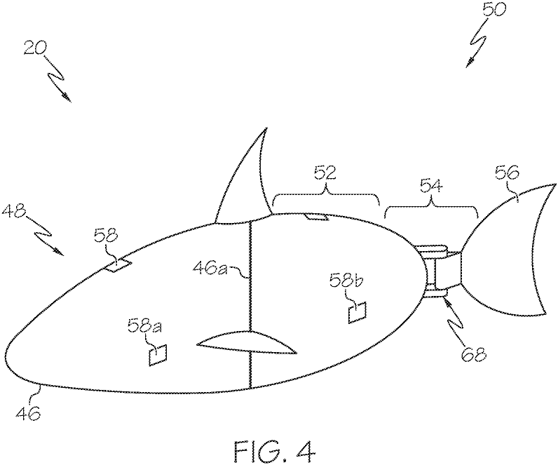

FIGS. 4-7 illustrate aspects of an exemplary autonomous underwater vehicle 20 in the form of a bio-inspired robotic fish. A robotic fish that can mimic the undulatory locomotion of a biological fish, producing oscillating foil propulsion, can generate thrust more efficiently than standard marine propellers.

AUV 20 has a body 46 with a forward section 48 and an aft or tail section 50, separated for example proximate to a mid-point 46a of the body. In the robotic fish embodiments, tail section 50 includes an anterior section 52, peduncle 54, and a tail fin 56 (caudal fin). AUV 20 includes a sensor 58 to measure an attribute in a water column that is indicative of carbon dioxide concentration, for example pH or carbon dioxide partial pressure. In an exemplary embodiment, the AUV includes two types of sensors, a first sensor type 58a located on forward section 48 and a second sensor type 58b on tail section 50. One of first sensor type 58a and second sensor type 58b to measure pH and the other one of the first and the second sensor type to measure carbon dioxide partial pressure.

Body 46 is formed in the shape of a fish, such as a tuna. Tail section 50 is moveable relative to forward section 48 to produce an undulatory body wave and produce an oscillating foil propulsion 55. The undulatory body wave is laterally along the longitudinal axis “X” of body 46.

The propulsion system 60 to produce the undulatory body wave and oscillating foil propulsion has a direct current (DC) motor as a main actuator 62. A double-slider crank 64 is designed for one motor 62 to drive two joints 66, 68. The double-slider crank 64 converts a rotary motion into two flapping motions that can motivate the tail section to realize oscillating foil flapping. The double-slider crank is designed to act as a peduncle and tail and thus joints 66, 68 provide the flapping motion of peduncle 54 and tail fin 56.

A three-joint robotic fish with a hybrid propulsion system can produce a bias angle for maneuvering and/or produce three-joint flapping. An example of a three-joint propulsion system includes a servomotor 70, main actuator 62, and double-slider crank 64 with a forward joint 72. Servomotor 70 is employed with forward joint 72 located forward of peduncle joint 66 and tail joint 68 to bias tail section 50 and enhance the turning maneuverability. Servomotor 70 and forward joint 72 generates a bias angle 74 (FIG. 5) inspired by large ocean fish species such as tuna and shark that turn by producing a bend anterior of the peduncle. In an exemplary operation, servomotor 70 in front of the peduncle outputs a constant angle and does not participate in the flapping. In some operations, the servomotor may be operated to produce three-joint flapping. The speed and steering are independently controlled by the DC motor and the servomotor, respectively.

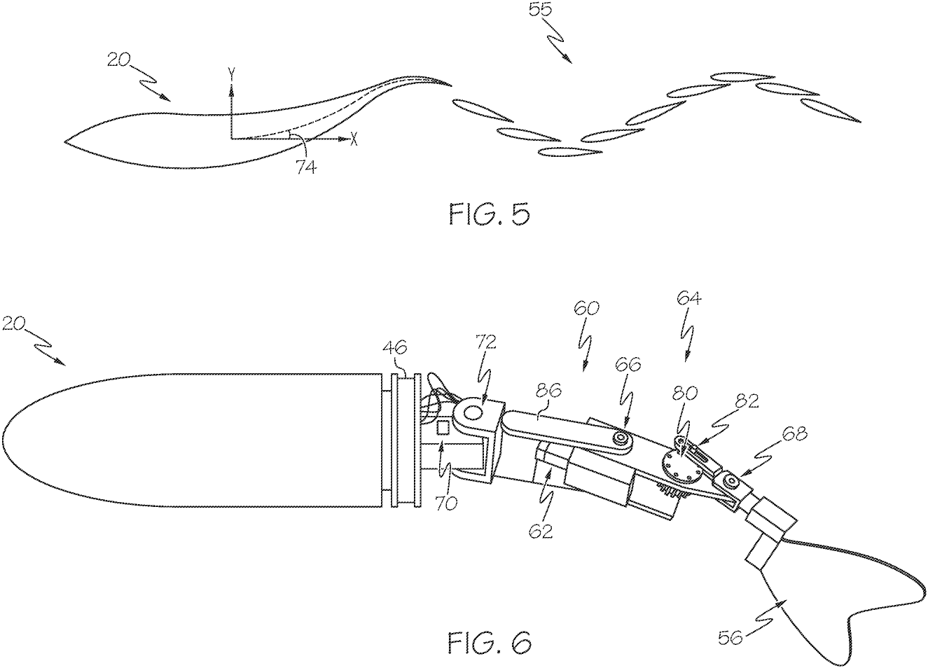

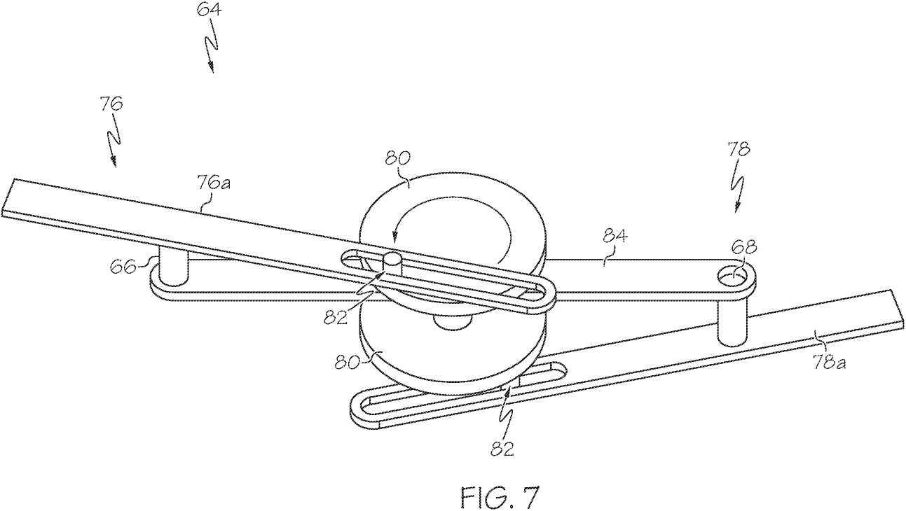

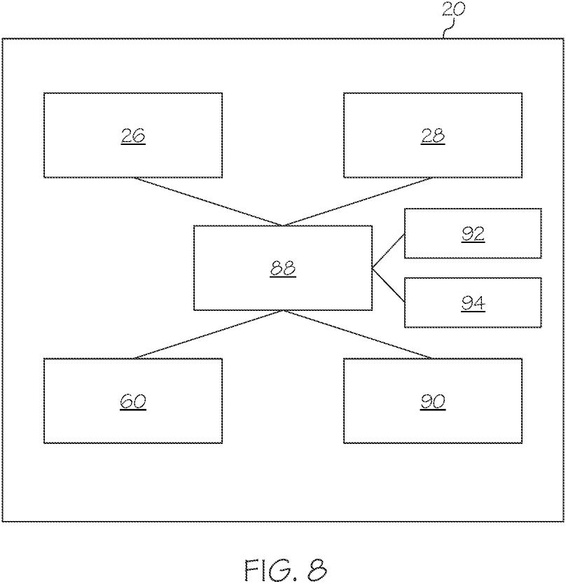

FIG. 7 schematically illustrates joints 66, 68 of double-slider-crank 64 in FIG. 6. Double-slider-crank 64 is a two-segment mechanism that produces a constant phase shift in a rotary axis to realize an oscillating foil. It includes two slider-crank mechanisms 76, 78 driven by a pair of rotatory uni-axial plates 80. Each slider-crank mechanism 76, 78 has a respective slider 76a, 78a that is pin-slot 82 mated with a respective plate 80. Main actuator 62 (FIG. 6) drives the rotatory plates 80. A support bar 84 is connected to joints 66, 68. Slider 76a is connected to the body of the robotic fish and is in a fixed position. Slider 78a connects to tail fin 56. Plates 80 and support bar 84 are the first segment that oscillates about joint 66 and slider 78a and the tail fin oscillate about joint 68. Referring back to FIG. 6, in the hybrid propulsion system, forward joint 72 is coupled via arm 86 to peduncle joint 66 to produce a bias angle for steering.

FIG. 8 is a schematic illustration of an autonomous underwater vehicle 20. AUV 20 includes an onboard controller 88 connected to one or more transmitters 26, a propulsion system 60, one or more sensors 58, and buoyancy control 90. Controller 88 includes a processor and instructions to perform the tasks of AUV 20. For example, controller 88 includes control logic 92 to implement a carbon dioxide gradient estimation and calculate gradient vectors. Controller 88 may include swarm logic 94 to guide the AUVs 20 using the gradient vector to a highest carbon dioxide concentration. The swarm logic may be distributed to the AUVs.

Buoyancy control system 90 may take various configurations. In a non-limiting example, buoyancy control system 90 includes a reversible fuel cell, a gas bladder in communication with the reversible fuel cell, and circuitry e.g., controller, to operate the reversible fuel cell in an electrolyzer mode and a fuel cell mode. A regenerative or reversible fuel cell (RFC) is a device that can operate alternatively as an electrolyser and as a fuel cell. When the fuel cell is a polymer electrolyte membrane (PEM) fuel cell, the RFC is based on a low-temperature water-splitting reaction. In an electrolyser mode of a PEM RFC, liquid water is split into hydrogen and oxygen gas by electrical energy input into the PEM RFC to increase buoyancy. In fuel cell mode, the PEM RFC regenerates electricity and water using the stored hydrogen and oxygen thereby reducing buoyancy of the vehicle.

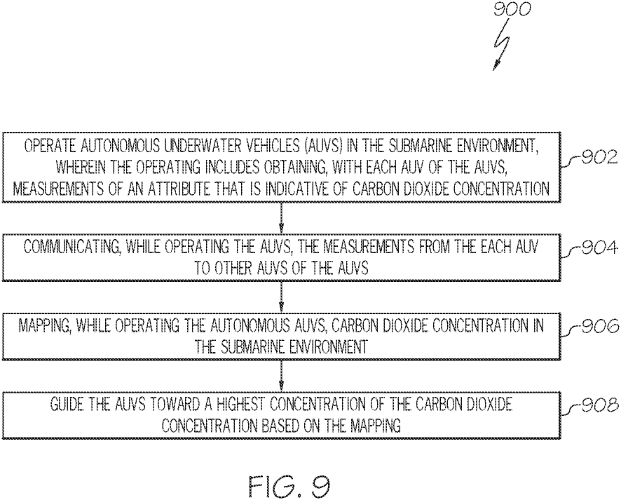

FIG. 9 is a block diagram of an exemplary method 900 for detecting and locating a carbon dioxide leak in a submarine environment. FIG. 9 is described with reference to FIGS. 1-8. At block 902, autonomous underwater vehicles are operated in a submarine environment where each AUV of the AUVs obtains measurements of an attribute that is indicative of carbon dioxide concentration. For example, the attribute may be pH and/or carbon dioxide partial pressure. The AUVs may be assigned to travel in a defined area so that the plurality of AUVs monitor a large area in the submarine environment. At block 904, each AUV communicates the measurements with the other AUVs. At block 906, while operating the carbon dioxide concentration in the submarine environment is mapped. Each AUV may perform the mapping or mapping may be performed remotely and communicated to each of the AUVs. The mapping includes implementing a carbon dioxide gradient estimation using the measurements and calculating a gradient vector based on the carbon dioxide gradient estimation. At block 908, the AUVs are guided toward the highest concentration of the mapped carbon dioxide concentration.

Although relative terms such as “outer,” “inner,” “upper,” “lower,” and similar terms have been used herein to describe a spatial relationship of one element to another, it is understood that these terms are intended to encompass different orientations of the various elements and components in addition to the orientation depicted in the figures. Furthermore, as used herein, the terms “connect,” “connection,” “connected,” “in connection with,” and “connecting” may be used to mean in direct connection with or in connection with via one or more elements. Similarly, the terms “couple,” “coupling,” and “coupled” may be used to mean directly coupled or coupled via one or more elements. The terms “substantially,” “approximately,” “generally,” and “about” are defined as largely but not necessarily wholly what is specified (and includes what is specified; e.g., substantially 90 degrees includes 90 degrees and substantially parallel includes parallel), as understood by a person of ordinary skill in the art. The extent to which the description may vary will depend on how great a change can be instituted and still have a person of ordinary skill in the art recognized the modified feature as still having the required characteristics and capabilities of the unmodified feature.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a,” “an” and other singular terms are intended to include the plural forms thereof unless specifically excluded.

What is claimed is:

1. A method for detecting and locating a carbon dioxide leak in a submarine environment, the method comprising:
operating autonomous underwater vehicles (AUVs) in the submarine environment, wherein the operating includes obtaining, with each AUV of the AUVs, measurements of an attribute indicative of carbon dioxide;
communicating, while operating the AUVs, the measurements from the each AUV to other AUVs of the AUVs;
mapping, while operating the autonomous AUVs, carbon dioxide concentration in the submarine environment; and
guiding the AUVs toward a highest concentration of the carbon dioxide concentration based on the mapping.

2. The method of claim 1, wherein the guiding the AUVs toward the highest concentration is in response to the measurements being indicative of the carbon dioxide leaking into a water column of the submarine environment.

3. The method of claim 1, wherein the each AUV performs the mapping.

4. The method of claim 1, wherein the mapping comprises implementing a carbon dioxide gradient estimation using the measurements and calculating a gradient vector based on the carbon dioxide gradient estimation.

5. The method of claim 1, wherein the mapping the carbon dioxide concentration comprises mapping at least one of acidity or carbon dioxide partial pressure.

6. The method of claim 1, wherein the each AUV is a robotic fish having a forward section and a tail section with a tail fin, wherein the operating the AUVs comprises manipulating the at least two joints in the tail section to produce oscillating foil propulsion.

7. The method of claim 1, wherein the attribute comprises acidity and carbon dioxide partial pressure.

8. The method of claim 1, wherein the communicating the measurements from the each AUV to the other AUVs is via magnetic induction communication.

9. The method of claim 1, wherein the operating and the mapping is performed proximate a carbon dioxide pipeline.

10. The method of claim 1, wherein:
the operating and the mapping is performed proximate a carbon dioxide pipeline;
the mapping comprises implementing a carbon dioxide gradient estimation using the measurements and calculating a gradient vector based on the carbon dioxide gradient estimation; and
the guiding the AUVs toward the highest concentration is in response to the measurements being indicative of carbon dioxide leaking into a water column of the submarine environment.

11. The method of claim 10, wherein the mapping is performed by the each AUV.

12. A system for detecting and locating a carbon dioxide leak in a submarine environment, the system comprising:
autonomous underwater vehicles (AUVs), each AUV of the AUVs comprising:
a sensor to obtain measurements of an attribute in the submarine environment that is indicative of carbon dioxide;
a transmitter to communicate the measurements to the each AUV of the AUVs; and
a control logic to implement a carbon dioxide gradient estimation using the measurements to calculate a gradient vector; and
a swarm logic to guide the AUVs using the gradient vector to a highest carbon dioxide concentration.

13. The system of claim 12, wherein the attribute is at least one of acidity or carbon dioxide partial pressure.

14. The system of claim 12, wherein the sensor comprises a pH senor and a carbon dioxide partial pressure sensor.

15. The system of claim 12, wherein the each AUV is a robotic fish comprising a forward section and a tail section with a tail fin and a motor operable to move the tail section to produce an oscillating foil propulsion.

16. The system of claim 12, wherein the each AUV is a robotic fish comprises:
a forward section and a tail section with a tail fin;
a steering actuator operable to bias the tail section relative to the forward section; and
a main actuator operable to move at least two joint in the tail section to produce an oscillating foil propulsion.

17. The system of claim 12, wherein the transmitter is a magnetic induction device.

18. The system of claim 12, wherein the swarm logic is distributed to the AUVs.

19. The system of claim 12, wherein:
the each AUV is a robotic fish comprises a forward section and a tail section with a tail fin, a steering actuator operable to bias the tail section relative to the forward section, and a main actuator operable to move at least two joints in the tail fin to produce an oscillating foil propulsion; and
the sensor comprise a first sensor located on the forward section and a second sensor located on the tail section.

20. The system of claim 19, wherein one of the first sensor and the second sensor measures acidity and the other one of the first sensor and the second sensor measures carbon dioxide partial pressure.
